(19) United States
(12) Patent Application Publication
(10) Pub. No.: US 2018/0313842 A1
(43) Pub. Date: Nov. 1, 2018

(54) METHOD FOR MONITORING EFFICACY OF A CANCER THERAPY USING CIRCULATING TUMOR CELLS AS A BIOMARKER

(71) Applicants: THE BOARD OF TRUSTEES OF THE UNIVERSITY OF ILLINOIS, Urbana, IL (US); THE UNIVERSITY OF NORTH CAROLINA AT CHAPEL HILL, Chapel Hill, NC (US)

(72) Inventors: Andrew Wang, Durham, NC (US); Michael Eblan, Durham, NC (US); Seungpyo Hong, Naperville, IL (US); Ja Hye Myung, Chicago, IL (US)

(21) Appl. No.: 15/565,728

(22) PCT Filed: May 12, 2016
(86) PCT No.: PCT/US16/31982

§ 371 Date: Oct. 11, 2017

Related U.S. Application Data
(60) Provisional application No. 62/161,595, filed on May 14, 2015.

(51) Int. Cl. G01N 33/574; A61N 5/10; G01N 33/50; G01N 33/569

(52) U.S. Cl. CPC G01N 33/57488; A61N 5/1064; G01N 33/5008; G01N 2800/7028; A61N 5/1001; G01N 2800/52; G01N 33/56966

(57) ABSTRACT

Methods for monitoring efficacy of cancer therapies, e.g., radiation therapy, using circulating tumor cell kinetics as a predictive marker are described.

### INTRODUCTION

This application claims benefit of priority to U.S. Provisional Patent Application Ser. No. 62/161,595, filed May 14, 2015, the content of which is incorporated herein by reference in its entirety.

This invention was made with government support under contract number R01-CA182528 awarded by the National Institutes of Health and contract number DMR-1409161 awarded by the National Science Foundation. The government has certain rights in the invention.

### BACKGROUND

Circulating tumor cells (CTCs) are an important biomarker in cancer management. Its established clinical application includes the use as a non-invasive “liquid biopsy” of the tumor and as a prognostic biomarker in breast, prostate and colorectal cancers (Cohen, et al. (2008) J. Clin. Oncol. 26:3213-3221; Cristofanilli, et al. (2004) N. Engl. J. Med. 351:781-791; de Bono, et al. (2008) Clin. Cancer Res. 14:6302-6309), as well as an efficacy marker in prostate cancer (de Bono, et al. (2008) Clin. Cancer Res. 14:6302-6309; Goldkorn, et al. (2014) J. Clin. Oncol. 32:1136-1142; Sher, et al. (2011) J. Clin. Oncol. 29 (suppl; abstract LBA4517):293s; Lowes, et al. (2012) Clin. Transl. Oncol. 14:150-156). However, the relatively low sensitivity of existing CTC assays has limited its wide clinical adoption. CTCs are extremely rare, composed of as few as one in a billion hematological cells in the blood. Moreover, the majority of CTCs in the bloodstream undergo apoptosis or necrosis during circulation, resulting in an even lower number of detectable CTCs in peripheral blood. To overcome the rarity of CTCs for their use as a biomarker, the development of devices that can detect and capture CTCs with high sensitivity and specificity is critical to CTC research and clinical translation.

A myriad of CTC detection methods have been developed. CELLSEARCH™, the only FDA-approved system to date, and most of the currently available CTC detection technologies utilize immunoaffinity-based enrichment depending on the expression of tumor epithelial markers, such as epithelial cell adhesion molecule (EpCAM). However, the EpCAM-based CTC detection technologies have been shown to have low sensitivity, as many CTCs frequently display down-regulated epithelial markers on the cell surface primarily due to epithelial mesenchymal transition (EMT). Moreover, the typically low capture purity (a low percentage of CTCs among all captured cells) reported using the existing detection methods hinders post-capture analysis of CTCs.

Several strategies have been shown to improve CTC detection. First, surface functionalization with aEpCAM and E-selectin exhibits greater capture efficiency (by up to 3.2-fold) compared to surface with aEpCAM only. This enhancement is attributed to E-selected-induced cell rolling efficiently recruiting fast-flowing cells in a flow chamber onto the capture surface (Myung, et al. (2010) Langmuir 26:8589-96). Further, it has been demonstrated that multivalent binding can improve the capture capability of the surface. The capture capability of the surface is significantly improved with the utilization of G7 poly(amidoamine) (PAMAM) dendrimer-mediated multivalent binding effect, as observed by an over 1 million-fold enhancement in dissociation constant and an over 7-fold increase in capture efficiency (Myung, et al. (2011) Angew Chem. Int. Ed. Engl. 50(49):11769-72). In addition, it has been shown that the combination effect of cell rolling and multivalent binding could be applied with multiple cancer cell-specific antibodies, such as aEpCAM, anti-human epidermal growth factor receptor-2 (aHER-2), and anti-epidermal growth factor receptor (aEGFR) (Myung, et al. (2014) Anal. Chem. 86(12):6088-94). Utilizing these strategies, a CTC device designated UICHIP™ has been developed for efficient capture of CTCs, hypothesizing that the observed enhancement is applicable for clinical CTCs (WO 2010/124227 and WO 2015/134972).

### SUMMARY OF THE INVENTION

This invention is a method for monitoring efficacy of a cancer therapy by (a) determining the number of circulating tumor cells (CTCs) in a biological sample (e.g., peripheral blood) from a subject before a cancer therapy, and (b) comparing the number of CTCs determined in (a) to a number of CTCs determined from a similar biological sample from the same subject at one or more time points during or after the cancer therapy, wherein the number of CTCs is determined using a flow-based device having at least one chamber comprising an immobilized cell-rolling agent (e.g., E-selectin) and one or more immobilized CTC-specific capturing agents (e.g., antibodies that bind epithelial cell adhesion molecule (EpCAM), epidermal growth factor receptor-2 (HER-2), and epidermal growth factor receptor (EGFR)). In one embodiment, a change in the number of CTCs (e.g., a decrease or increase) during or after treatment with the cancer therapy is indicative of the subject's response to the cancer therapy. In another embodiment, the one or more CTC-specific capturing agents are immobilized via a modified poly(amidoamine) dendrimer covalently attached to polyethylene glycol. In further embodiments, the cancer therapy is for treatment of a head and neck cancer, lung cancer, rectal cancer, esophageal cancer or cervical cancer. In a particular embodiment, the cancer therapy is radiation therapy and the method further includes the step of (c) modifying the radiation therapy (e.g., increasing or decreasing ionizing radiation dose, administering the radiation by hypofractionation or hyperfractionation, or administering a chemotherapy, gene therapy, immunotherapy, targeted therapy, hormonal therapy, radiosensitizer or a combination thereof) if the number of the CTCs changes during or after the radiation therapy. In specific embodiments, the flow-based device has a detection threshold of about 2.1 cells per mL and provides CTC purity levels of approximately 49%.

### BRIEF DESCRIPTION OF THE DRAWINGS

FIGS. 1A-1C show enhanced CTC capture sensitivity through a combination of dendrimers and multiple antibodies on UICHIP™-S. FIG. 1A, Significant CTC counts per mL blood from all patients (01-21) obtained using UICHIP™-S. FIG. 1B, Significantly higher CTC counts on UICHIP™-S per 7.5 mL of patients' blood captured, compared to the CELLSEARCH™ results from the literature (5±2, n=19, CELLSEARCH™ vs. 1663±389, n=20). The average lines indicate the mean±SE. FIG. 1C, Fold enhancement of antibody mixture (ABMIX), G7 dendrimers (G7), and combination of the two, relative to the CTC counts captured on the control surface coated with aEpCAM only (dotted line). The average lines indicate the mean±SE.

FIGS. 2A-2C show enhanced CTC capture specificity via addition of E-selectin-mediated cell rolling to UICHIP™-D. FIG. 2A, Significant CTC counts per mL blood from patients (UNC 02-21) obtained using UICHIP™-D. Note that the CTC count of patient 01 was not included because the blood sample was treated with EDTA, instead of heparin, destabilizing the rolling response of the cells. FIG. 2B, Comparison of the CTC counts measured using UICHIP™-D and UICHIP™-S. FIG. 2C, CTC counts obtained using blood samples from healthy donors. The baseline CTC counts using UICHIP™-S and UICHIP™-D were measured at 7.7±1.1 and 2.1±0.3 cells per mL, respectively. FIG. 2D, Significantly enhanced CTC capture purities (%) among all captured cells using UICHIP™-D, compared to those using UICHIP™-S. This result indicates that the capture specificity of UICHIP™-D was dramatically enhanced via E-selectin-mediated cell rolling.

FIGS. 3A and 3B show the therapeutic effect of monitoring radiotherapy (RT) using UICHIP™-D. FIG. 3A, Compared to the reported CTC counts in HNSCC cancer patients using CELLSEARCH™ (Gröbe, et al. (2014) Clin. Cancer Res. 20:525-33; Bozec, et al. (2013) Eur. Arch. Otorhinolaryngol. 270:2745-9; Grisanti, et al. (2014) PLoS ONE 9(8):e103918; Nichols, et al. (2012) Head Neck 34:1440-4), significantly higher numbers of CTCs were captured using UICHIP™-D (1663.3±389.2 cells/7.5 mL of blood, mean±standard error (SE)) were detected. FIG. 3B, In the 16 patients with complete CTC measurements during the course of RT, the CTC counts at the Pre-RT (median 152 cells/mL, ranging from 43 to 849 cells/mL) were statistically significantly decreased in response to RT (median 29 cells/mL at the End-RT, range of 2 to 150 cells/mL, p=0.001).

### DETAILED DESCRIPTION OF THE INVENTION

Circulating tumor cells (CTCs) are an important biomarker in cancer care. However, the clinical utilization of CTCs has been limited by the low sensitivity of existing CTC capture assays. It has now been found that, using the UICHIP™ CTC capture platform, capture efficiencies of various tumor cells is significantly improved through a combination of efficient recruitment of flowing cells to the surface by E-selectin-mediated cell rolling, strong surface binding of tumor cells by poly(amidoamine) (PAMAM) dendrimer-mediated multivalent binding effect, and a mixture of multiple cancer cell-specific antibodies such as aEpCAM, aHER-2, and aEGFR. In particular, it was observed that the high sensitivity of UICHIP™, largely owing to dendrimer-mediated multivalent binding effect of multiple antibody mixtures, enabled the capture of CTCs ranging from 18.5 to 662 CTCs per mL. The induction of cell rolling on UICHIP™-D significantly improved the CTC detection specificity (up to 48.6% purity). Importantly, based on the changes of CTC counts among Pre- and End-radiotherapy, UICHIP™-D showed that CTCs could be used as a predicative biomarker for treatment response. Accordingly, the UICHIP™-D capture of CTCs is of use in monitoring therapeutic effect and cancer progression and in allowing post-capture analysis of the isolated CTCs. For example, gene sequences related to cancer development (e.g., KRAS and EGFR) could be assessed in patient-derived CTCs isolated using UICHIP™-D thereby facilitating the discovery of new cancer biomarkers and ultimately personalized medicine applications.

Thus, the present invention relates generally to assays to detect cancer and predict its progression in conjunction with cancer therapies. In some cases, where patients are suspected to be at risk of cancer, prophylactic treatments may be employed. In other cancer subjects, diagnosis may permit early therapeutic intervention. In yet other situations, the result of the assays described herein may provide useful information regarding the need for repeated treatments. Finally, the present invention is useful in demonstrating therapeutic efficacy, e.g., monitoring treatment and assessing which therapies do and do not provide benefit to a particular patient.

In accordance with the method of this invention, the number of CTCs in a biological sample from a subject are determined before a cancer therapy commences, and compared with the number of CTCs in a similar biological sample from the same subject at one or more time points during or after the therapy. In particular embodiments, the method can further include treating the cancer based on whether the level of CTCs is high. Successful treatment of a cancer is evident when the subject receives a therapeutic benefit from the cancer therapy. Such benefit includes a decrease in the number of CTCs present in the biological sample after treatment as compared to before treatment with the therapy. Additional indicators of successful treatment can include a reduction in the frequency or severity of the signs or symptoms of the subject's cancer, an improvement in well-being and/or an increase in survival.

The term “circulating tumor cell” or “CTC” is intended to mean any circulating cancer cell that is found in a sample obtained from a subject. Typically, CTCs have been shed from a solid tumor. As such, OTCs are often epithelial cells shed from solid tumors that are found in very low concentrations in the circulation of patients with cancers. CTCs may also be mesothelial cells from sarcomas or melanocytes from melanomas.

As used herein, the term “biological sample” refers to any sample that includes CTCs. Sources of samples include whole blood, bone marrow, pleural fluid, peritoneal fluid, central spinal fluid, metastasis, fresh biopsy samples (e.g., fresh prostate biopsy sample), urine, saliva and bronchial washes. In particular, the sample is a blood sample, including, for example, whole blood or any fraction or component thereof. A blood sample, suitable for use with the present invention may be extracted from any source known that includes blood cells or components thereof, such as veinous blood, arterial blood, peripheral blood, tissue, cord blood, and the like. For example, a sample may be obtained and processed using well-known and routine clinical methods (e.g., procedures for drawing and processing whole blood). In a particular embodiment, a sample may be peripheral blood drawn from a subject with cancer.

A subject with cancer is intended to refer to any individual or patient from whom CTCs (or a sample containing CTCs) is obtained or to whom the subject methods are performed. Generally the subject is human, although the subject may be an animal, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas).

In certain embodiments, the subject has cancer, is suspected of having cancer or is a risk of having cancer (e.g., based upon family history, predisposition or exposure to a carcinogen). Such a cancer can include cancer of the lung, breast, colon, prostate, pancreas, esophagus, all gastro-intestinal tumors, urogenital tumors, kidney cancers, melanomas, endocrine tumors, sarcomas, etc. In particular embodiments, the cancer is breast, cervical, endometrial, prostate, lung, pancreatic, liver, gastrointestinal, colorectal, or head and neck cancer. In particular embodiments, the subject has a solid tumor. In one embodiment, the cancer is head and neck cancer. In another embodiment, the cancer is lung cancer (small and non-small cell). In a further embodiment, the cancer is rectal cancer. In yet another embodiment, the cancer is esophageal cancer. In a still further embodiment, the cancer is cervical cancer.

Cancer therapies or treatments that can be monitored using the method of this invention include, but not limited to, chemotherapy, radiotherapy, surgery, gene therapy, immunotherapy, targeted therapy, hormonal therapy or a combination thereof. In certain embodiments, the cancer therapy being monitored is a radiotherapy. In some embodiments, the radiotherapy is used in conjunction with a chemotherapy.

Chemotherapy. A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Chemotherapeutic agents include, but are not limited to, paclitaxel (taxol); docetaxel; germicitibine; Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfanoral; calusterone; capecitabine; carboplatin; carmustine; carmustine with Polifeprosan Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); mechlorethamine (nitrogenmustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and any mixtures thereof.

Radiotherapy. Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiation therapy used according to the present invention may include, but is not limited to, the use of y-rays, X-rays, and/or the directed delivery of radioisotopes to tumor cells. Other forms of DNA damaging factors are also contemplated such as microwaves and UV-irradiation. Dosage ranges for X-rays range from daily doses of 50 to 200 roentgens for prolonged periods of time (3 to 4 weeks) , to single doses of 2000 to 6000 roentgens. Dosage ranges for radioisotopes vary widely, and depend on the half-life of the isotope, the strength and type of radiation emitted, and the uptake by the neoplastic cells. It is further contemplated that radiotherapy may include the use of radiolabeled antibodies to deliver doses of radiation directly to the cancer site (radioimmunotherapy) and/or include the use of a radiosensitizer.

Immunotherapy. In the context of cancer treatment, immunotherapeutics, generally, rely on the use of immune effector cells and molecules to target and destroy cancer cells. Trastuzumab (HERCEPTIN™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Examples of immunotherapies currently under investigation or in use are immune adjuvants, e.g., Mycobacterium bovis, Plasmodium falciparum, dinitrochlorobenzene and aromatic compounds (U.S. Pat. No. 5,801,005 and U.S. Pat. No. 5,739,169); cytokine therapy, e.g., interferons β, and γ; IL-i, GM-CSF and TNF; gene therapy, e.g., TNF, IL-1, IL-2, p53 (U.S. Pat. No. 5,830,880 and U.S. Pat. No. 5,846,945); and monoclonal antibodies, e.g., anti-ganglioside GM2, anti-HER-2, anti-p185 (U.S. Pat. No. 5,824,311).

Surgery. Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

Other Agents. Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

Hormonal therapy may also be used. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

The amount of therapeutic agent to be applied in the method set forth herein will be whatever amount is pharmaceutically effective and will depend upon a number of factors, including the identity and potency of the chosen therapeutic agent. One of ordinary skill in the art would be familiar with factors that are involved in determining a therapeutically effective dose of a particular agent. The therapeutic agent may be applied once or more than once. In non-limiting examples, the therapeutic agent is applied once a day, twice a day, three times a day, four times a day, six times a day, every two hours when awake, every four hours, every other day, once a week, and so forth. Treatment may be continued for any duration of time as determined by those of ordinary skill in the art.

The results presented herein demonstrate that CTCs are a predicative biomarker for cancer therapy. More specifically, CTC kinetics, i.e., the change in CTC numbers over a cancer therapy treatment regime, is used in assessing complete or incomplete response to a cancer therapy. According the method of this invention, the number of CTCs is determined both before and after treatment and can also optionally be determined during a course of treatment to assess the efficacy of the cancer therapy regime, e.g., timing and/or dose. Changes in CTC numbers or CTC kinetics are evaluated by comparing the number of CTCs before treatment to the number of CTCs during and/or after treating. In one embodiment, the consistent decrease of CTC over the treatment course predicts complete tumor response to the therapy (e.g., radiotherapy with or without chemotherapy). In another embodiment, if the CTC numbers increase during treatment, regardless of the final CTC count, then the CTC kinetic/biomarker predicts for incomplete response to the therapy.

As demonstrated herein, the biomimetic platform, UICHIP™, provided a high degree of sensitivity and specificity for measuring CTCs. Indeed, regardless of cancer stage, patient's medical history, or cancer type, UICHIP™-D was able to capture a mean and median of 222 and 101 CTCs per mL of peripheral blood, respectively. This was substantially higher than the cutoff values of 7.5 CTCs (UICHIP™-S) and 2.1 CTCs (UICHIP™-D) per mL of blood donated from the healthy volunteers. Accordingly, the numbers of CTCs in the method of this invention are determined using a UICHIP™ device, which has a cell rolling-inducing agent and at least one CTC-specific capturing agent attached to a substrate. See WO 2010/124227 and WO 2015/134972. Specifically, the method of this invention uses a flow-based device wherein the device includes at least one chamber having an immobilized cell-rolling agent and at least one immobilized CTC-specific capturing agent. In some embodiments, the capturing agent is an antibody, an antibody fragment, an engineered antibody, folic acid, transferrin, a peptide, and an aptamer that binds a moiety on the surface of a CTC. In particular embodiments, the flow-based device includes capturing agents that bind to one or more of epithelial cell adhesion molecule (EpCAM), human epidermal growth factor receptor-2 (HER-2), epidermal growth factor receptor (EGFR), carcinoembryonic antigen (CEA), Prostate specific antigen (PSA), CD24, and folate binding receptor (FAR). To facilitate CTC capture, the capturing agents are immobilized via attachment to a surface of the device via a modified poly(amidoamine) dendrimer covalently attached to polyethylene glycol. In other embodiments, the cell rolling-inducing agent is a selectin or a CTC binding fragment of a selectin. More particularly, the selectin is E-selectin, P-selectin or L-selectin. Advantageously, the flow-based device used in the method of this invention provides efficient recruitment of flowing cells to the surface by selectin-mediated cell rolling; strong surface binding of tumor cells by poly(amidoamine) dendrimer-mediated multivalent binding effect; and the use of multiple cancer cell-specific antibodies, e.g., aEpCAM, aHER-2, and aEGFR. Moreover, using the flow-based device, a detection threshold of about 2.1 cells per mL could be achieved and CTC purity was approximately 49% compared to a device without a cell-rolling agent (typically 0.04%-10.7%). Accordingly, the method of this invention also provides for a detection threshold of about 2.0 cells per mL and CTC purity levels of at least about 15%, 20%, 25%, 30%, 40%, 45% or 50%. Given the detection threshold and high level of purity attained using the method of this invention, any changes in CTC numbers or CTC kinetics during or after a cancer therapy can be readily measured.

Accordingly, the present method also provides for modifying the cancer therapy based upon a change in CTC numbers (i.e., an increase or decrease) after treatment. In particular, when the cancer therapy is radiation therapy, said therapy can be modified by increasing or decreasing the dose of ionizing radiation when the number of CTCs increase (i.e., an incomplete response) or decrease (i.e., a complete response), respectively. In other embodiments, hypofractionation or hyperfractionation of the dose of ionizing radiation is administered to the tumor. In further embodiments, the radiation therapy is modified by administering a chemotherapy, gene therapy, immunotherapy, targeted therapy, hormonal therapy, radiosensitizer or a combination thereof.

As a particular feature of the present invention is a method for monitoring efficacy of a radiation therapy by (a) determining the number of circulating tumor cells (CTCs) in a biological sample from a subject before a administering a dose of radiation therapy, and (b) comparing the number of CTCs determined in (a) to a number of CTCs determined from a similar biological sample from the same subject at one or more time points during or after the radiation therapy, wherein the number of CTCs is determined using a flow-based device having at least one chamber comprising an immobilized cell-rolling agent and one or more immobilized CTC-specific capturing agents. In one embodiment, the method further includes the step of administering an increased dose of radiation to the subject, where the dose of radiation is increased compared to the dose administered to a subject that does not have elevated levels of CTCs in the peripheral blood in response to starting radiation treatment. In accordance with this embodiment, the increased dose of radiation can be administered in a hyperfractionated or hypofractionated mode. In another embodiment, the method further includes the step of administering a decreased dose of radiation to the subject, where the dose of radiation is decreased compared to the dose administered to a subject that has elevated levels of CTCs in the peripheral blood in response to starting radiation treatment. In yet another embodiment, the method further includes the step of administering a dose of radiation to the subject that is similar to the dose administered to a subject that does not have elevated levels of CTCs in the peripheral blood, in combination with a pharmaceutically effective amount of a chemotherapy, gene therapy, immunotherapy, targeted therapy, hormonal therapy, radiosensitizer or a combination thereof.

The following non-limiting examples are provided to further illustrate the present invention.

### EXAMPLE 1

### Materials and Methods

Materials. Anti-human epithelial-cell-adhesion-molecule (EpCAM)/TROP1 antibody (aEpCAM), anti-human epidermal growth factor receptor-2 (HER-2)/TROP1 antibody (aHER-2), and recombinant human E-selectin (E-selectin) were purchased from R&D systems (Minneapolis, Minn.). Anti-human epidermal growth factor receptor (EGFR) antibody (aEGFR, N-20) was obtained from Santa Cruz Biotech (Dallas, Tex.). Epoxy-functionalized glass surfaces (SUPEREPOXY2®) were purchased from TeleChem International, Inc. (Sunnyvale, Calif.). PAMAM dendrimers (generation 7), bovine serum albumin (BSA), and all other chemicals, unless noted otherwise, were obtained from Sigma-Aldrich (St. Louis, Mo.) and used without further purification unless otherwise specified.

Surface Functionalization by Immobilization of Capture Agents. Surface functionalization was performed using established methods (Myung, et al. (2014) Anal. Chem. 86(12):6088-94; Myung, et al. (2011) Angew Chem. Int. Ed. Engl. 50(49):11769-72). Briefly, an epoxy-functionalized glass slide was first fitted with a polydimethylsiloxane (PDMS) gasket with patterns to define the area for immobilization of different agents. The surface was then functionalized by sequential immobilization of heterobifunctional PEG (NH₂-PEG-COOH), generation 7 partially carboxylated PAMAM dendrimers, and antibodies using EDC/NHS chemistry (Myung, et al. (2011) Angew Chem. Int. Ed. Engl. 50(49):11769-72). For antibody conjugation, all antibody solutions of aEpCAM, aHER-2, aEGFR were used at a final concentration of 5 μg/mL. The volume of each of the reagent solutions was fixed at 250 μL for UICHIP™-S (i.e., device without E-selectin) and 200 μL for UICHIP™-D (i.e., device with E-selectin). In the case of UICHIP™-D, whole antibody-immobilized surfaces were treated with 0.4 mL of E-selectin at a concentration of 5 μg/mL in phosphate-buffered saline (PBS) for 4 hours. All surface reactions were carried out at room temperature with constant gentle shaking, and between all preparation steps, the surfaces were washed with distilled de-ionized (DDI) water and PBS three times to remove the residual reagents. Potential non-specific binding of both protein-coated and uncoated regions was blocked by a final incubation with 1 μg/mL methoxy PEG-NH₂ (Nektar Therapeutics, Huntsville, Ala.) solution. The functionalized surfaces were kept at 4° C., and the experiments using the surfaces were performed within one week after the surface preparation.

Study Design. This was a single-institution prospective study conducted at the Lineberger Comprehensive Cancer Center at the University of North Carolina-Chapel Hill (UNC). Patients with histologically proven cancers were eligible. Radiologically confirmed stage II, III, or IV disease was required, and patients were to commence treatment with standard radiotherapy (RT) protocols, with or without chemotherapy. All patients gave written, informed consent to the IRB-approved study protocols. Data was collected for age, ethnicity, histological subtype, smoking status, sites of metastasis, RT received, survival, and RT response.

Blood Samples. Approximately 12 mL of whole peripheral blood was drawn from either healthy donors or cancer patients. The blood was collected into heparin-treated BD VACUTAINER tubes to prevent coagulation, except for the first patient enrolled, whose baseline specimen was collected into EDTA-treated BD VACUTAINER tubes. Blood specimens were drawn from cancer patients, kept at ambient temperature, and analyzed within 24 hours after blood collection. Mononuclear cells including CTCs in buffy coat were separated from whole blood using FICOLL-PAQUE Plus (Stemcell Technologies Inc., Vancouver, Canada) as previously described publication (Myung, et al. (2014) Anal. Chem. 86(12):6088-94). After washing the buffy coat twice with 2% FBS-containing PBS, the recovered cells were suspended in 0.2 mL of the complete DMEM medium and used for subsequent experiments.

CTC Capture Assay. To capture CTCs from blood specimens, the UICHIP™-S platforms were incubated with the suspension of mononuclear cells in buffy coat in an incubator. The recovered buffy coat suspension was divided into two: the first half was mixed with 650 μL of the complete DMEM medium for UICHIP™-S and the other half was directly used for UICHIP™-D. The surfaces were incubated with 250 μL of the cell suspension for 2 hours.

For UICHIP™-D, flow chamber experiments were performed as previously reported (Myung, et al. (2010) Langmuir 26:8589-96). Suspension of the isolated buffy coat was injected into a flow chamber, using a syringe pump (New Era pump Systems Inc., Farmingdale, N.Y.). The flow chamber composed of two channels (60 mm (L)×10 mm (W)×0.125 mm (D) for each channel) was connected with tubing for injection of the blood samples. The UICHIP™-D capture of the cells was continuously monitored under flow at 25 μL/min, corresponding to 0.22 dyn/cm² of shear stress. The surface was then washed using complete DMEM medium for 20 minutes and PBS for 15 minutes at 100 μL/min (0.88 dyn/cm²). The whole capture process was monitored using an OLYMPUS IX70 inverted microscope (Olympus America, Inc., Center Valley, Pa.), a 10× objective, and a CCD camera (QImaging Retiga 1300B, Olympus America, Inc.).

To identify CTCs among the surface-captured cells, a series of immunostaining assays were performed. After fixed with 4% paraformaldehyde for 15 minutes, all captured cells were treated with 0.2 w/v% TRITON X-100 (penetrating buffer) for 5-10 minutes to enhance the antibody penetration. To prevent non-specific binding, whole slides were treated with 2 w/v% BSA solution for 30 minutes before immunostaining. The cells were then sequentially stained with the following antibodies: (1) rabbit antibody against human cytokeratin (CK; 1:50, abcam), (2) ALEXAFLUOR 594-conjugated secondary antibody against anti-CK (1:100, Invitrogen), (3) rabbit antibody against human CD45 (1:500, BD bioscience), and (4) ALEXAFLUOR 488-conjugated secondary antibody against anti-CD45 (1:100, Invitrogen). The DAPI-included mounting media (VectaShield Laboratories, Inc., Burlingame, Calif.) was also used to stain the nuclei of mononuclear cells and prevent photo-bleaching during analysis. The slides were then sealed with cover glass and nail polish, and were stored at 4° C. The immunostained platforms were scanned using a ZEISS 701 confocal microscope equipped with a motorized stage and 20× objective, and a CCD camera. The number of CK+/CD45−/DAPI+ CTCs on the surfaces was counted, based on the images taken from independent observations/measurements using ImageJ (NIH).

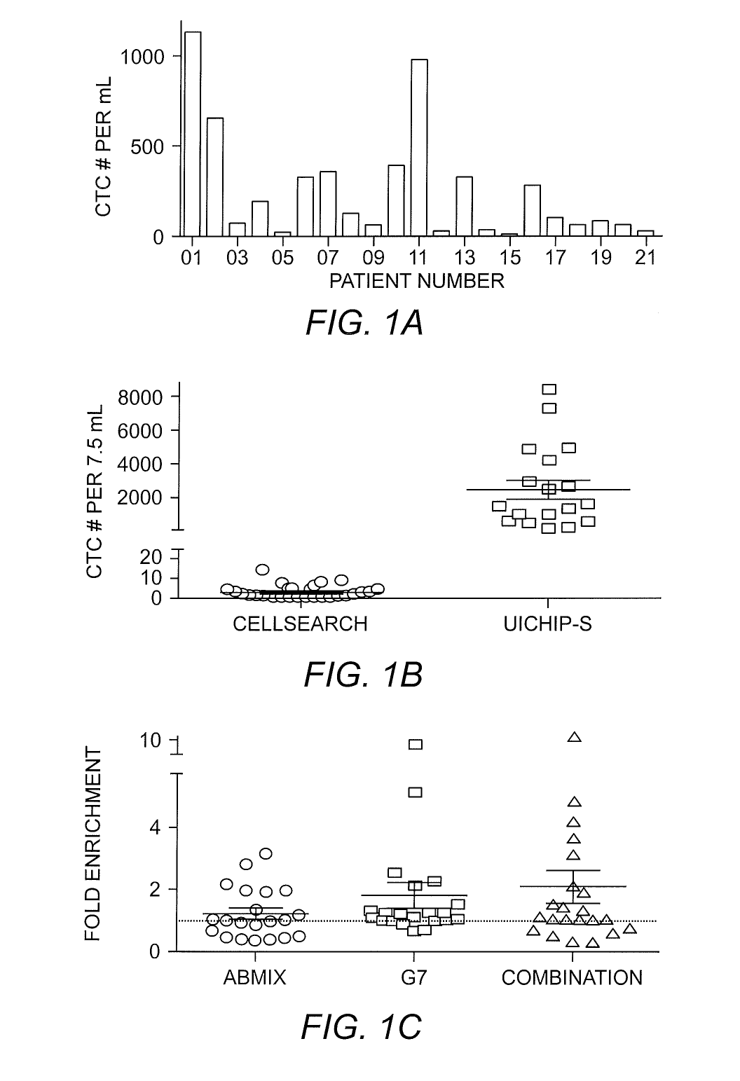

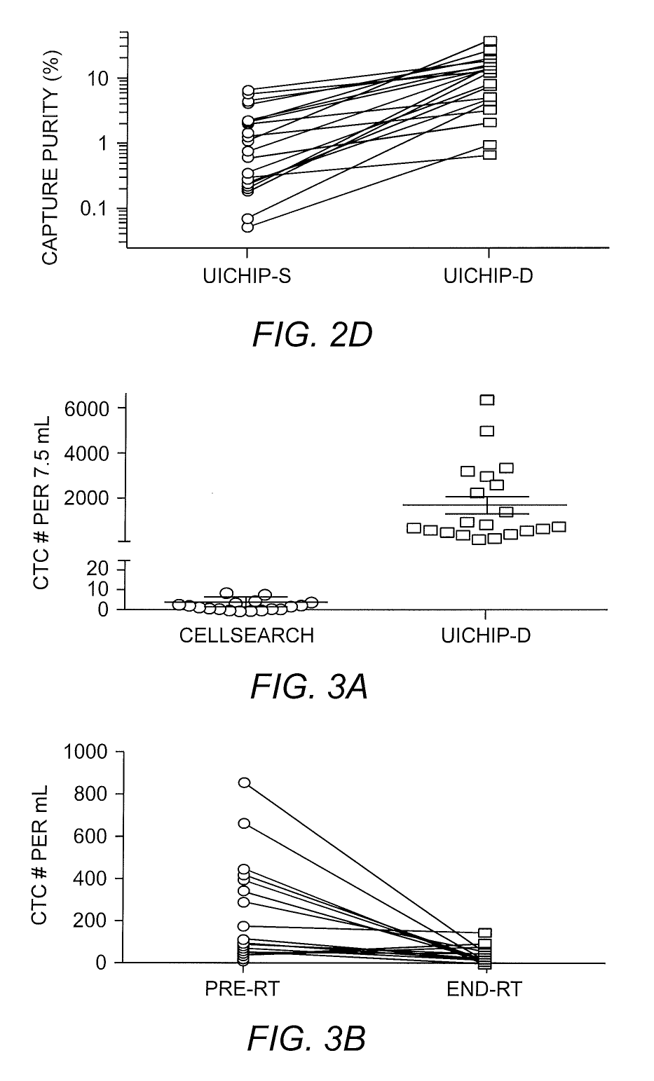

Statistical Analysis. The statistical analysis was performed using SPSS version 21.0 for WINDOWS (IBM Corp., Armonk, N.Y., USA). The difference in absolute CTC numbers between Pre-RT and End-RT for all patients was calculated using the Wilcoxon signed-rank test. The Friedman test was used to determine the statistical difference of absolute CTC levels obtained by the three different CTC capture platforms (PEG-aEpCAM, PEG-ABmix, and G7-ABmix) evaluated (data shown in FIG. 1C). The difference in absolute CTC numbers between the Pre-RT and End-RT was compared by the Wilcoxon signed-rank test (data shown in FIG. 3B). All statistical tests were performed at a significance level of P<0.05 (two-tailed).

### EXAMPLE 2

### Surface Preparation and UICHIP™ Fabrication

UICHIP™ integrating G7 PAMAM dendrimers, E-selectin, and antibody mixtures was fabricated using surface chemistries previously described (Myung, et al. (2011) Angew Chem. Int. Ed. Engl. 50(49):11769-72). Briefly, partially carboxylated G7 PAMAM dendrimers were immobilized on the epoxy-functionalized glass slides through a heterobifunctional polyethyleneglycol (PEG, COOH-PEG-NH₂) linker using 1-ethyl-3-(3-dimethylaminopropyl)carbodiimide/N-hydroxysulfosuccinimide) (EDC/NHS)-based amine-coupling chemistry (Myung, et al. (2011) Angew Chem. Int. Ed. Engl. 50(49):11769-72). Antibody mixtures (ABmix) of aEpCAM, aHER-2, and aEGFR were then conjugated to the carboxylate termini of G7 PAMAM dendrimers via EDC/NHS coupling (Myung, et al. (2011) Angew Chem. Int. Ed. Engl. 50(49):11769-72; Myung, et al. (2014) Anal. Chem. 86(12):6088-94). As this step allowed to consume most of the primary amine groups available on the dendrimer surface, it helped minimize non-specific, electrostatic interactions between positively charged amine termini of PAMAM dendrimers and negatively charged cell membranes. Compared to the surfaces without dendrimers, the PAMAM dendrimer-immobilized surfaces were able to immobilize a greater amount of antibodies due to their dendritic nanostructures, and mediated the multivalent binding effect to significantly enhance tumor cell binding. Human recombinant E-selectin molecules were additionally immobilized through forming covalent bonding between the amine groups of E-selectin and the epoxy groups on the glass slides to effectively recruit flowing cells to the capture surfaces. Finally, to minimize non-specific binding, the functionalized surfaces were incubated with methoxy-PEG-NH₂ to consume epoxy groups remaining on the surfaces. The surfaces were characterized using X-ray photoelectron spectroscopy and fluorescence microscopy to confirm the successful surface functionalization.

### EXAMPLE 3

### Patient Demographics

Patients with histologically confirmed primary carcinoma undergoing RT for oncologic management were enrolled into this study. A total of 21 patients with rectal (n=1), cervical (n=1), prostate (n=1), oral cavity (n=2), paranasal sinus (n=3), or oropharynx (n=13) cancers were recruited over a six month period. Patient demographic and clinical information is summarized in Table 1. Baseline blood specimens (Pre-RT) were collected within 1 week of starting RT, typically on the day of CT simulation for RT planning or on the day of pre-treatment patient set-up. During RT, specimens were collected at up to 3 time points, including during the first week of RT (1W-RT), mid-way through RT (Mid-RT), and during the last week of RT (End-RT). A final specimen was collected at least 4 weeks later than the last week of RT (Post-RT).

TABLE 1

Evaluable
Patients
(N = 21)

| | No. | % |
|---|---|---|
| Age at baseline, years | | |
| Median | | 57 |
| Range | | 42-84 |
| Gender | | |
| Female | 7 | 33 |
| Male | 14 | 67 |
| Race | | |
| Caucasian | 18 | 86 |
| African American | 3 | 14 |
| Cancer Type | | |
| Head and Neck | 18 | 86 |
| Oropharynx | 13 | 62 |
| Oral Cavity | 2 | 10 |
| Paranasal | 3 | 14 |
| Rectal | 1 | 5 |
| Cervical | 1 | 5 |
| Prostate | 1 | 5 |
| Tumor Stage at Diagnosis | | |
| II | 3 | 14 |
| III | 1 | 5 |
| IV | 17 | 81 |
| Histological Subtype | | |
| Squamous Cell Carcinoma | 18 | 86 |
| HPV/P16 Positive | 9 | 43 |
| HPV/P16 Negative | 4 | 19 |
| HPV/P16 Unknown | 2 | 10 |
| Adenocarcinoma | 1 | 5 |
| Sinonasal | 2 | 10 |
| Smoking Status | | |
| Current Smoker | 4 | 19 |
| Former Smoker | 8 | 38 |
| Never Smoker | 9 | 43 |

A total of 19 of 21 enrolled patients (90%) had at least one on-treatment specimen collected for subsequent CTC analysis after baseline measurement (Pre-RT), while 17 patients (81%) had a final blood draw prior to completion of RT (End-RT). Post-RT specimens were collected from a total of 13 patients (62%).

### EXAMPLE 4

### Enhanced CTC Detection Sensitivity Using Dendrimers and Multiple Antibodies

The CTC detection sensitivity of the surface with the multiple antibodies immobilized on the surfaces functionalized with G7 PAMAM dendrimers was measured using the clinical blood samples from those cancer patients. Note that the device that employed G7 dendrimers and ABmix to detect CTCs under static conditions (without flow) was indicated as UICHIP™-S. Standard immunostaining against cytokeratin (CK, epithelial marker), CD45 (leukocyte marker), and nuclei (DAPI) was performed to identify CK+/CD45−/DAPI+ CTCs among captured cells on the surface. As shown in FIG. 1A, UICHIP™-S surface captured CTCs from all patients with CTC counts ranging from 4 to 1,134 cells/mL. The CTC counts in head and neck squamous cell cancinoma (HNSCC) patients were then compared to the reported numbers obtained using CELLSEARCH™ (Gröbe, et al. (2014) Clin. Cancer Res. 20:525-33; Bozec, et al. (2013) Eur. Arch. Otorhinolaryngol. 270:2745-9; Grisanti, et al. (2014) PLoS ONE 9(8):e103918; Nichols, et al. (2012) Head Neck 34:1440-4) because HNSCC patients were the majority cancer patient population in this study. Note that the number of CTCs per mL in the results was multiplied by 7.5 to match the blood volume used for UICHIP™-S. FIG. 1B shows significantly higher numbers of CTCs captured using the present surface (2,448±569.4 cells/7.5 mL of blood, mean±standard error (SE)), as compared to the reported results of CELLSEARCH™, where only a few CTCs in 7.5 mL were detected. The effect of the individual components on capture efficiency was also investigated, by separately counting CTCs using surfaces with: (1) PEG-aEpCAM; (2) PEG-ABmix; and (3) G7-ABmix conjugates (UICHIP™-S). A pair-wise comparison of each treatment provided insight into the contribution of each surface component to the overall enhancement of CTC capture sensitivity: a pair of (1) and (2) for ABmix effect; another pair of (2) and (3) for G7 PAMAM dendrimer effect; and the third pair of (1) and (3) for the combined effect of the ABmix and G7 PAMAM dendrimers. As shown in FIG. 1C, the results of these comparisons, each with a >1 fold-enhancement, indicated that there was a positive contribution by each of the particular surface components. The percentages of the samples that exhibited positive contributions (≥1 fold-enhancement) via the three comparisons (ABmix, G7 dendrimer, and the combination) were 57.1%, 81.0%, and 76.2%, respectively (FIG. 1C).

### Example 5: Enhanced CTC Detection Using UICHIP™-D

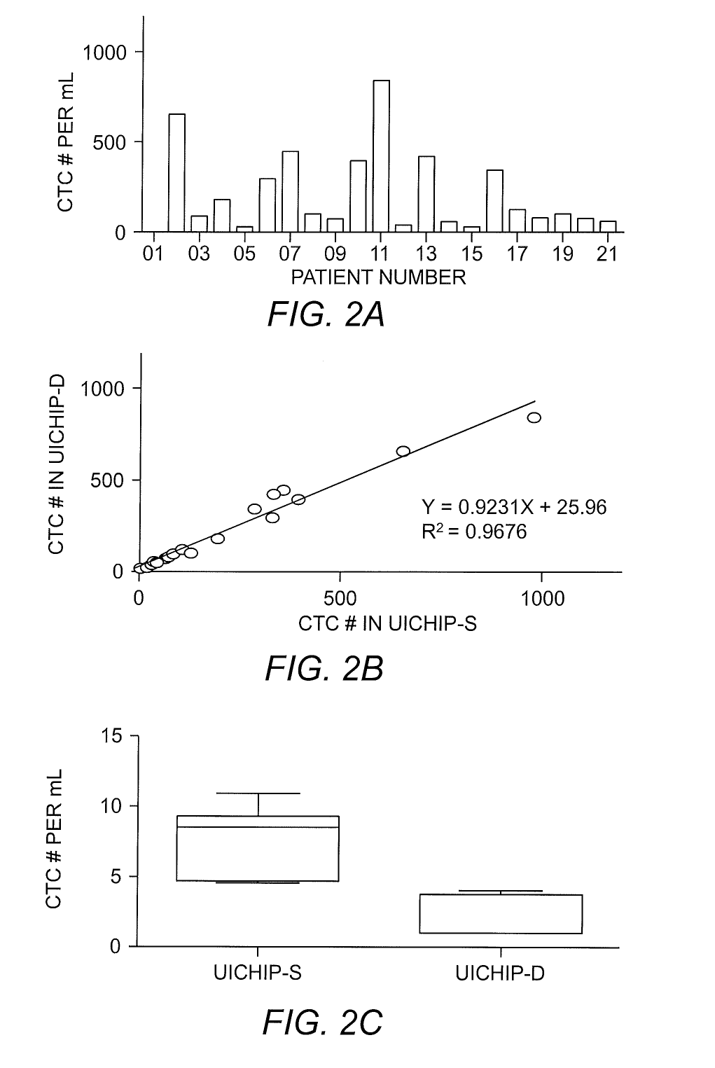

CTC detection specificity was significantly enhanced by E-selectin-mediated cell recruitment under flow. UICHIP™ that integrates the ABmix, G7 dendrimers, and E-selectin (denoted as UICHIP™-D) successfully captured CTCs in a custom-prepared flow chamber from the blood samples of the 20 patients, and the numbers of CTCs varied in a range of 19 to 662 cells per mL (FIG. 2A). Due to the fact that Ca⁺⁺-dependent cell rolling with E-selectin on UICHIP™-D does not occur in the presence of EDTA (a Ca⁺⁺ chelating agent), the CTC count for EDTA-treated patient sample was excluded for analysis using UICHIP™-D. The CTC counts obtained using UICHIP™-D were similar to those of UICHIP™-S (R²=0.9676, FIG. 2B), which indicated that the detection sensitivity of UICHIP™ was not significantly affected by cell rolling. Using blood specimens from three healthy participants without cancer history, the CTC counts of UICHIP™-S and of UICHIP™-D were 7.7±1.1 and 2.1±0.3 cells per mL, respectively, and were used to establish the detection thresholds (FIG. 2C). Despite no significant improvement in sensitivity, cell rolling induced by E-selectin of UICHIP™-D notably enhanced the capture purity, compared to UICHIP™-S (FIG. 2D). The capture purity (specificity) was calculated by the ratios of the CK+/CD45−/DAPI+ CTC counts per total DAPI+ cells including leukocytes and CTCs. The specificity of UICHIP™-D in terms of CTC purity among captured cells (up to 48.6%) was dramatically improved by up to 93.5-fold, compared to that of UICHIP™-S (typically 0.04%-10.7%). The fluorescence images after immunostaining clearly showed the difference between the absence (UICHIP™-S) and the presence of E-selectin (UICHIP™-D), i.e., significantly reduced non-specific capture of leukocytes.

### Example 6

### Analytical Significance of UICHIP™-D for CTC Detection

The surfaces for CTC detection were further compared in terms of CTC counts in the blood samples from the 20 patients measured at the Pre-RT versus those from the 16 patients measured at the End-RT. At the Pre-RT, there was a significant increase in CTC counts with the G7-ABmix surface compared to the PEG-aEpCAM (p=0.043) and the PEG-ABmix (p<0.001) surface platforms, respectively. This difference in CTC counts between different surface preparations persisted when CTC levels were measured at the End-RT (G7-ABmix versus PEG-aEpCAM, p=0.006; G7-ABmix versus PEG-ABmix, p=0.001). However, there was no statistically significant difference in CTC counts when comparing the PEG-aEpCAM and the PEG-ABmix methods at the Pre-RT (p=0.906) and End-RT (p=0.076). A comparison of absolute CTC numbers obtained by all three methods demonstrated significantly higher CTC capture for the G7-ABmix platform UICHIP™-D, compared to the PEG-aEpCAM and PEG-ABmix surfaces (p<0.001). UICHIP™-D with G7-ABmix and E-selectin captured on average 222 CTCs/mL (range, 19-849) at the Pre-RT, and 44 CTCs/mL (range, 2-150) at the End-RT. It was significantly more CTCs captured when compared to both the PEG-aEpCAM and PEG-ABmix methods, where only 187 CTCs/mL (range, 9-814) and 161 CTCs/mL (range, 16-511) at the Pre-RT and 32 CTCs/mL (range, 2-201) and 33 CTCs/mL (range, 1-131) at the End-RT were collected, respectively.

### Example 7

### Clinical Significance of CTC Counts Using UiChip™-D

Of the total population, all 20 patients (100%) examined with UICHIP™-D had detectable CTCs in their blood at the Pre-RT, at an average of 222 CTCs per mL and median count of 101 CTCs per mL (range, 19 to 662 cells per mL). It was significantly higher than 2.1±0.3 (average±S.E., FIG. 2C) CTCs in 1.0 mL of blood found in the samples from three healthy donors (p=0.0091). Importantly, the CTC counts measured using UICHIP™-D were significantly higher than the reported CTC counts (139-6364 cells/7.5 mL of blood) in HNSCC patients using CELLSEARCH™ (Grobe, et al. (2014) Clin. Cancer Res. 20:525-33; Bozec, et al. (2013) Eur. Arch. Otorhinolaryngol. 270:2745-9; Grisanti, et al. (2014) PLoS ONE 9(8):e103918; Nichols, et al. (2012) Head Neck 34:1440-4), as shown in FIG. 3A. Interestingly, from the 17 patients with complete CTC measurements during the course of RT, a statistically significant reduction in CTC counts upon RT was observed. The average CTC count decreased from 222 cells/mL (range of 19 to 849 cells/mL) at the Pre-RT, to 44 cells/mL (range of 2 to 150 cells/mL) at the End-RT (p=0.001, FIG. 3B).

What is claimed is:

1. A method for monitoring efficacy of a cancer therapy compromising
(a) determining the number of circulating tumor cells (CTCs) in a biological sample from a subject before a cancer therapy, and
(b) comparing the number of CTCs determined in (a) to a number of CTCs determined from a similar biological sample from the same subject at one or more time points during or after the cancer therapy, wherein the number of CTCs is determined using a flow-based device having at least one chamber comprising an immobilized cell-rolling agent and one or more immobilized CTC-specific capturing agents.

2. The method of claim 1, wherein a change in the number of CTCs during or after treatment with the cancer therapy is indicative of the subject's response to the cancer therapy.

3. The method of claim 2, wherein the change is an increase.

4. The method of claim 2, wherein the change is a decrease.

5. The method of claim 1, wherein the cell-rolling agent is E-selectin.

6. The method of claim 1, wherein the one or more immobilized CTC-specific capturing agents comprise antibodies that bind epithelial cell adhesion molecule (EpCAM), epidermal growth factor receptor-2 (HER-2), and epidermal growth factor receptor (EGFR).

7. The method of claim 6, wherein the one or more CTC-specific capturing agents are immobilized via a modified poly(amidoamine) dendrimer covalently attached to polyethylene glycol.

8. The method of claim 1, wherein the biological sample is peripheral blood.

9. The method of claim 1, wherein the cancer therapy is for treatment of a solid tumor.

10. The method of claim 1, wherein the cancer therapy is for treatment of a head and neck cancer, lung cancer, rectal cancer, esophageal cancer or cervical cancer.

11. The method of claim 1, wherein the cancer therapy comprises radiation therapy.

12. The method of claim 11, further comprising (c) modifying the radiation therapy if the number of the CTCs changes during or after the radiation therapy.

13. The method of claim 12, wherein the radiation therapy is modified by increasing ionizing radiation dose.

14. The method of claim 13, wherein the radiation therapy is modified by decreasing ionizing radiation dose.

15. The method of claim 13, wherein the radiation therapy is modified by hypofractionation.

16. The method of claim 13, wherein the radiation therapy is modified by hyperfractionation.

17. The method of claim 13, wherein the radiation therapy is modified by administering a chemotherapy, gene therapy, immunotherapy, targeted therapy, hormonal therapy, radiosensitizer or a combination thereof.

18. The method of claim 1, wherein the flow-based device comprises a detection threshold of about 2.1 cells per mL.

19. The method of claim 1, wherein purity of the CTCs is approximately 49%.

20. A method for monitoring efficacy of a radiation therapy comprising
(a) determining the number of circulating tumor cells (CTCs) in a biological sample from a subject before a administering a dose of radiation therapy, and
(b) comparing the number of CTCs determined in (a) to a number of CTCs determined from a similar biological sample from the same subject at one or more time points during or after the radiation therapy, wherein the number of CTCs is determined using a flow-based device having at least one chamber comprising an immobilized cell-rolling agent and one or more immobilized CTC-specific capturing agents.
